(19) United States
(12) Patent Application Publication
(10) Pub. No.: US 2025/0107861 A1
(43) Pub. Date: Apr. 3, 2025

(54) STEERABLE LASER ABLATION THERAPY ROBOT

(71) Applicant: THE REGENTS OF THE UNIVERSITY OF CALIFORNIA, Oakland, CA (US)

(72) Inventors: Jun Sheng, Eastvale, CA (US); Saeed Rezaeian, Riverside, CA (US)

(21) Appl. No.: 18/899,942

(22) Filed: Sep. 27, 2024

Related U.S. Application Data
(60) Provisional application No. 63/541,504, filed on Sep. 29, 2023.

(51) Int. Cl. A61B 34/00; A61B 17/00; A61B 18/00; A61B 18/24; A61B 34/30; A61B 90/00; A61M 25/01; A61M 25/06

(52) U.S. Cl. CPC A61B 34/71; A61B 18/24; A61B 2017/00323; A61B 2017/00336; A61B 2017/00991; A61B 2018/00577; A61B 2034/301; A61B 2090/064; A61M 25/0113; A61M 25/0147; A61M 2025/0681

(57) ABSTRACT

A steerable laser ablation therapy robot for use in minimally invasive neurosurgery is disclosed. The steerable robotic device comprises a rigid straight outer tube; at least one telescopic flexible tendon-driven inner tube, including an outer telescopic flexible tendon-driven inner tube; and an optical fiber extending through the at least one telescopic flexible tendon-driven inner tube, the optical fiber coupled distally to a distal tip of the outer telescopic flexible tendon-driven inner tube and proximally to a laser generator.

### REFERENCE TO RELATED APPLICATIONS

This application claims priority to U.S. Provisional Application No. 63/541,504 that was filed on Sep. 29, 2023. The entire content of the application referenced above is hereby incorporated by reference herein.

### GOVERNMENT FUNDING

This invention was made with government support under R21 EB033896 awarded by the National Institute of Health, National Institute of Biomedical Imaging and Bioengineering. The government has certain rights in the invention.

### TECHNICAL FIELD

This document pertains generally, but not by way of limitation, to a miniaturized steerable robot that can navigate inside the human body along curved trajectories and provide laser ablation therapy.

### BACKGROUND

Minimally invasive neurosurgery (MIN) is a field that encompasses the use of minimally invasive techniques in performing surgical procedures on the brain and spinal cord. MIN aims to improve accuracy and precision, promote faster recovery time, reduce the physical and emotional stress of patients, and minimize scarring and long-term complications. MIN has been extensively researched for a variety of significant neurological disorders, such as brain tumors, intracerebral hemorrhage (ICH), and spinal disorders. For instance, ablation therapy (e.g., laser ablation) has emerged as a minimally invasive technique for initial and recurrent brain tumors. Neurosurgeons insert and rotate a straight rigid probe along a straight path and deliver ablation energy to different directions at different locations. Nevertheless, current ablation probes cannot effectively treat tumors that are large, have irregular shapes, and/or have multiple foci. Multiple insertions may be needed, potentially leading to increased trauma to the patient.

Thus, there is a need for improved MIN technology and techniques to safely and effectively treat diseased tissue at multiple locations while minimizing collateral damage to healthy tissue.

### SUMMARY

Certain embodiments provide a steerable robotic device comprising: a rigid straight outer tube; at least one telescopic flexible tendon-driven inner tube, including an outermost telescopic flexible tendon-driven inner tube; and an optical fiber extending through the at least one telescopic flexible tendon-driven inner tube, the optical fiber coupled distally to a distal tip of the outermost telescopic flexible tendon-driven inner tube and proximally to a laser generator.

In certain embodiments, the at least one telescopic flexible tendon-driven inner tube comprises a distal segment having notches extending longitudinally along a portion of a wall of the inner tube.

In certain embodiments, the at least one telescopic flexible tendon-driven inner tube is configured to bend along a curved trajectory.

In certain embodiments, the steerable robotic device further comprises a flexible sleeve covering the outermost telescopic flexible tendon-driven inner tube.

In certain embodiments, the rigid straight outer tube and the at least one telescopic flexible tendon-driven inner tube comprise superelastic nitinol.

In certain embodiments, the steerable robotic device further comprises a plurality of nonmagnetic rotary or linear actuators.

In certain embodiments, the steerable robotic device comprises an inner telescopic flexible tendon-driven inner tube, wherein a distal tip of the inner flexible tendon-driven inner tube is configured to remain inside the outermost telescopic flexible tendon-driven inner tube.

In certain embodiments, the steerable robotic device further comprises a processor configured to determine a deployment force of the at least one telescopic flexible tendon-driven inner tube based on a reference trajectory of the at least one telescopic flexible tendon-driven inner tube.

In certain embodiments, the processor is configured to determine the deployment force of the at least one telescopic flexible tendon-driven inner tube using a database of measured curvatures corresponding to one or more robot segment lengths.

In certain embodiments, the database of measured curvatures is generated based a measured soft-tissue deployment force of the one or more robot segment lengths.

In certain embodiments, the processor performs linear interpolation among the determined deployment forces to compute a tendon wire force for each telescopic flexible tendon-driven inner tube.

In certain embodiments, the rigid straight outer tube has an outer diameter of about 2.2 mm or smaller.

In certain embodiments, the steerable robotic device further comprises a FBG optical fiber extending through the at least one telescopic flexible tendon-driven inner tube, the FBG optical fiber including Fiber Bragg gratings (FBGs).

In certain embodiments, the FBG optical fiber including FBGs is embedded in a silicone polymer cylinder hosting photothermal nanoparticles, the silicone polymer cylinder located within the distal tip of the outer telescopic flexible tendon-driven inner tube.

In certain embodiments, the FBG optical fiber including FBGs is surrounded by a flexible saline cooling tube extending through the at least one telescopic flexible tendon-driven inner tube.

In certain embodiments, the FBGs is configured to monitor temperatures changes at the distal tip of the outer telescopic flexible tendon-driven inner tube.

Certain embodiments provide a method of ablating a target tissue using the steerable robotic device of claim 1, the method comprising: inserting the rigid straight outer tube into the target tissue; rotating the at least one telescopic flexible tendon-driven inner tube to define a first navigation plane; deploying the at least one telescopic flexible tendon-driven inner tube; adjusting a tendon wire to control a curved trajectory of the at least one telescopic flexible tendon-driven inner tube; ablating the target tissue; and retracting the at least one telescopic flexible tendon-driven inner tube.

In certain embodiments, the steerable robotic device comprises an inner telescopic flexible tendon-driven inner tube positioned inside the outer telescopic flexible tendon-driven inner tube, and the deploying step comprises: distally advancing the inner and outer telescopic flexible tendon-driven inner tubes together; and while holding the inner telescopic flexible tendon-driven inner tube in place, distally advancing the outer flexible tendon-driven inner tube.

In certain embodiments, the retracting step comprises: proximally retracting the outer telescopic flexible tendon-driven inner tube onto the inner telescopic flexible tendon-driven inner tube; and after the outer telescopic flexible tendon-driven inner tube is retracted onto the inner telescopic flexible tendon-driven inner tube, proximally retracting the inner and outer telescopic flexible tendon-driven inner tubes into the rigid straight outer tube.

In certain embodiments, the method further comprises rotating the at least one telescopic flexible tendon-driven inner tube to define a second navigation plane.

This summary is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes or subscripts may represent different instances of similar components. The drawings illustrate generally, by way of example, but not by way of limitation, various embodiments discussed in the present document.

FIG. 1 is an exploded side view of various components of a steerable robotic device in accordance with the present disclosure.

FIG. 2A is a side view a first embodiment of a steerable laser robot design.

FIG. 2B is a cross-sectional view along line 2b-2b shown in FIG. 2A.

FIG. 2C is an enlarged view of the inner tendon-driven inner tube tip region shown in FIG. 2B.

FIG. 2D is an enlarged view of the outer tendon-driven inner tube tip region shown in FIG. 2B.

FIG. 3A is a side view a second embodiment of a steerable laser robot design.

FIG. 3B is a cross-sectional view along line 3b-3b shown in FIG. 3A.

FIG. 3C is an enlarged view of the inner tendon-driven inner tube tip region shown in FIG. 3B.

FIG. 3D is an enlarged view of the outer tendon-driven inner tube tip region shown in FIG. 3B.

FIG. 4 is a schematic view of process for multi-site tumor ablation using the steerable robotic device.

### DETAILED DESCRIPTION

In the following description, for purposes of explanation, various details are set forth in order to provide a thorough understanding of some example embodiments. It will be apparent, however, to one skilled in the art that the present subject matter may be practiced without these specific details, or with slight alterations.

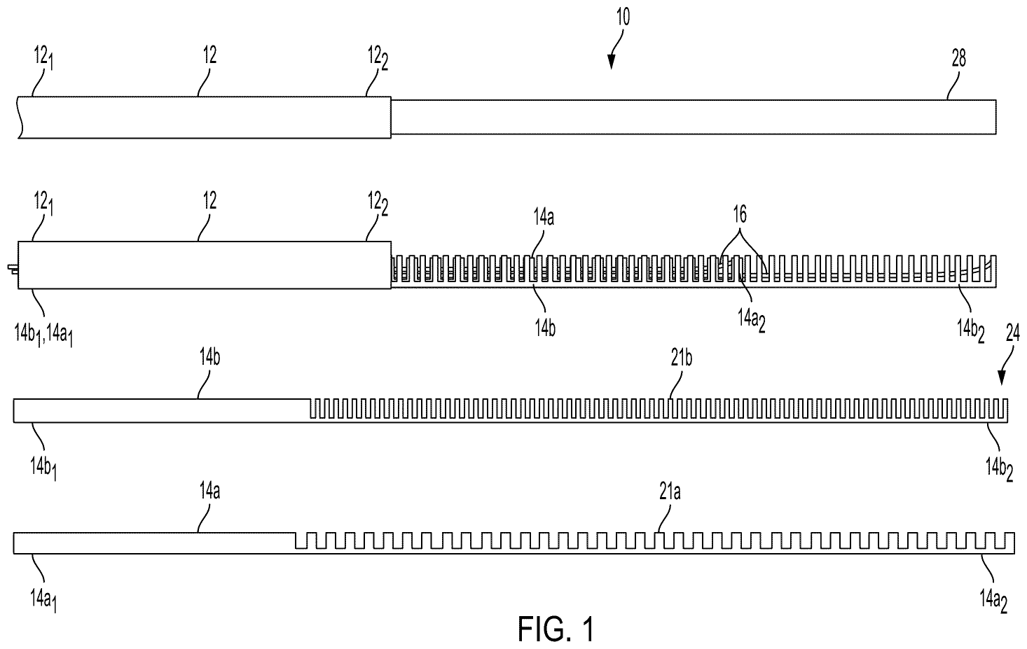

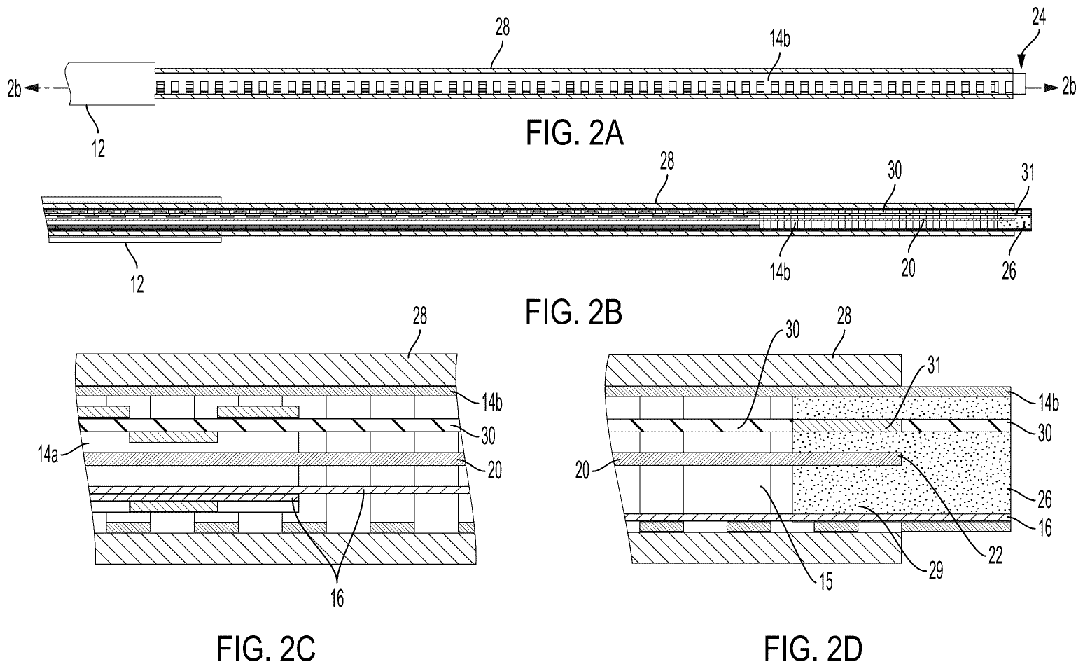

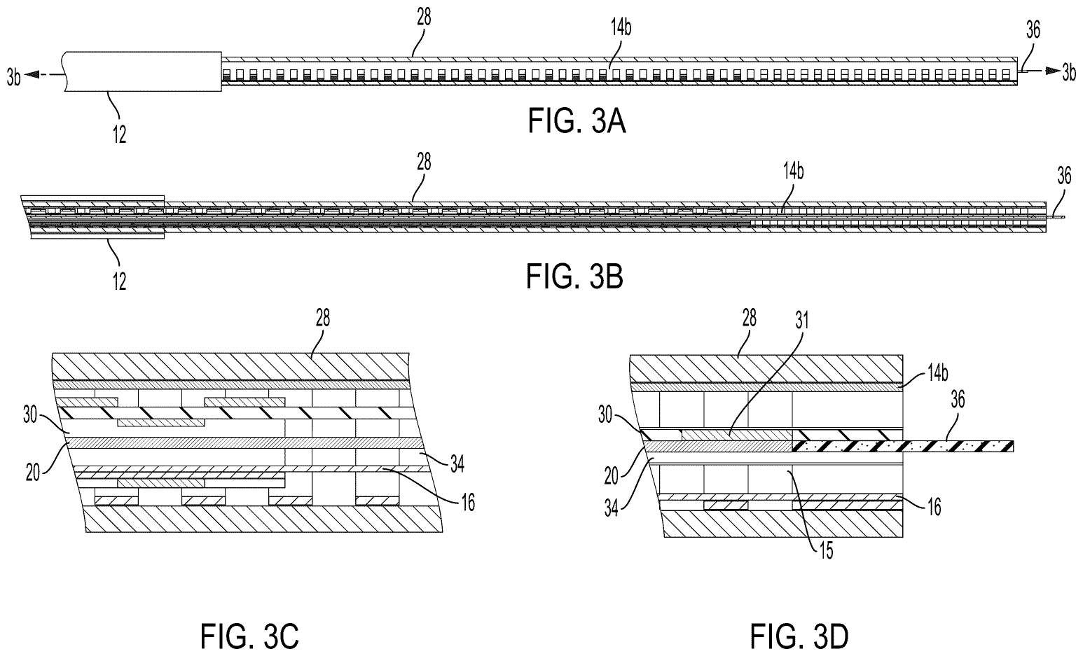

Referring to FIG. 1, a robotic device (or robot) 10 comprises at least two telescopic tubes—one rigid straight outer tube 12, having a proximal end 121 and a distal end 122, and one or more telescopic flexible tendon-driven inner tubes 14. In the example shown in FIG. 1, there is an inner tendon-driven inner tube 14a and an outer tendon-driven inner tube 14b, each of the tubes having a proximal end 14a1 and 14b1, a distal end 14a2 and 14b2. The robot device further includes a central lumen or channel 15 (shown in FIGS. 2C, 2D, 3C, and 3D). The outer diameter of the rigid straight outer tube 12 can be about 2.2 mm or smaller. The outer diameter of the telescopic flexible tendon-driven inner tubes 14 can be about 1.15 mm or smaller. The diameters of the rigid straight outer tube 12 and the telescopic flexible tendon-driven inner tubes 14 may both be much smaller than the corresponding diameters of existing robots used in MIN. A superelastic nitinol wire (also referred to as a “tendon wire”) 16 is fixed at the distal end of each tendon-driven inner tube 14, passed through the innermost channel of the robot 10, and connected proximally to a nonmagnetic actuation system (not shown). The nonmagnetic actuation system can include a plurality of nonmagnetic rotary or linear actuators, transmissions, and structures configured to rotate and translate the proximal end of each tendon-driven inner tube 14, as well at the proximal end of the rigid straight outer tube 12, as known to those skilled in the art. At least one optical fiber 20, shown in FIGS. 2C and 2D, extends through the one or more tendon-driven inner tubes 14. The optical fiber 20 includes a distal end 22 coupled to the distal tip 24 of the outermost tendon-driven inner tube 14 and proximally to a laser generator (not shown). The optical fiber 20 runs through the innermost channel of the robot 10.

As shown in FIG. 1, each tendon-driven inner tube 14 has unilateral notches 21a,b along at least a distal portion of its length so that the tube is flexible in its bending plane. When there is more than one tendon-driven inner tube 14, the inner tendon-driven inner tube 14a stays fully inside the adjacent outer tendon-driven inner tube 14b (namely, the tip of the outer tube 14b is extends further distally than the tip of the inner tube 14a) for smooth translation. A flexible sleeve 28 covers the outer tendon-driven inner tube 14b and extends distally to or near the proximal end 29 of a cylinder 26 made of a silicone polymer material, such as polydimethylsiloxane (PDMS), the cylinder 26 configured to host photothermal nanomaterials (e.g., nanoparticles) (shown in FIGS. 2B and 2D). It should be noted that although an inner tendon-driven inner tube 14a and an outer tendon-driven inner tube 14b are discussed in this example and other examples described herein, there could be more than two tendon-driven inner tubes 14, in which case the flexible sleeve 28 would cover the outermost of the tendon-driven inner tubes 14 (i.e., the outer tendon-driven inner tube 14b refers to the “outermost” tendon-driven inner tube in examples with more than two tendon-driven inner tubes). All the tubes, including the rigid straight outer tube 12 and the one or more telescopic flexible tendon-driven inner tubes 14, are made of superelastic nitinol so that the robot 10 is MRI compatible and intra-operative MRI can be used to guide the surgery and measure temperature change.

As shown in FIGS. 2B and 2D, the distal tip 24 of the outer tendon-driven inner tube 14b includes cylinder 26. The distal end 22 of the optical fiber 20 is embedded in the cylinder 26. Laser energy supplied through the optical fiber 20 via the cylinder 26 increases the temperature of the cylinder 26, thus ablating the nearby tissue around the tip of the robot 10. The robot 10 can also include at least one FBG optical fiber 30 with Fiber Bragg gratings (FBGs) 31 extending through the tendon-driven inner tubes 14a and 14b. The FBGs are located within the FBG optical fiber 30 at or near a distal tip section 32 of the outer tendon-driven inner tube 14b, also embedded within the cylinder 26. The FBG optical fiber 30 is proximally coupled to an optoelectronic interrogator (not shown). The FBGs can monitor temperature changes in the cylinder 26 to ensure the cylinder 26 and optical fibers 20 and 30 do not overheat or burn. Light inside the FBG optical fiber 30 is reflected by the FBGs to the interrogator. The wavelength of the reflected light changes during tissue ablation due to temperature change in the cylinder 26. The change in wavelength is measured by the optoelectric interrogator and converted to temperature change measurements, as explained, for example, by N. Hirayama and Y. Sano, “Fiber Bragg grating temperature sensor for practical use,” ISA Transactions, vol. 39, pp. 169-173, April 2000, which is hereby incorporated by reference in its entirety.

In an embodiment, shown in FIGS. 3C and 3D, the robot 10 includes a flexible cooling tube 34, which is proximally connected to a pump containing cooling fluid or saline (not shown) and runs through the innermost channel of the robot 10 to the distal tip 24 of the outer tendon-driven inner tube 14b. The optical fiber 20 providing laser ablation runs through the cooling tube 34 and is spliced at its distal end 22 with an optical diffusing tip 36. By supplying laser ablation through the optical fiber 20 to the optical diffusing tip 36, the laser will be diffused into tissue surrounding the distal tip 24 and thus ablate the tissue. Meanwhile, cooling saline is supplied through the flexible cooling tube 34 to avoid overheating and improve tissue ablation. The FBG optical fiber 30 including FBGs also runs through the cooling tube 34 and is bonded at its distal end to the assembly of the optical fiber 20 and diffusing tip 36. The FBGs can monitor the temperature change of the assembly to ensure the assembly does not overheat or burn.

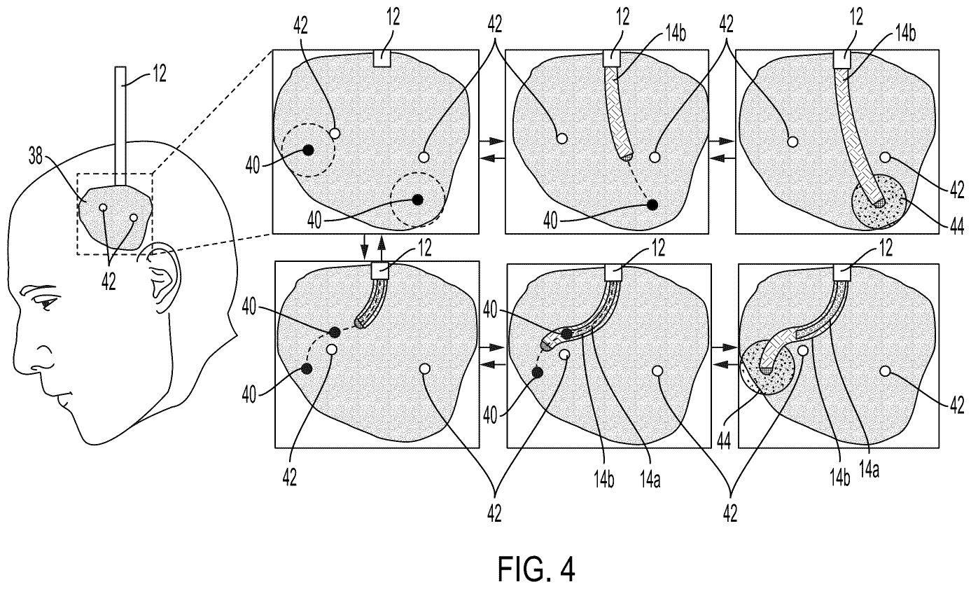

FIG. 4 illustrates the working principle of multi-site tumor ablation using the robot 10. First, a user (e.g., a neurosurgeon) designs ablation target points in a three-dimensional space (e.g., a patient's tumor) based on imaging (e.g., MRI imaging). The robot, or a computer processor communicatively linked to the robot, then determines two-dimensional target planes based on each target point and the straight rigid outer tube 12. In each two-dimensional target plane, a curvilinear reference trajectory consisting of at least one arc is generated based on the positions of the distal tip of the rigid straight outer tube 12 and the target points, as well as locations of critical structures. Each flexible tendon-driven inner tube 14 can be rotated at its proximal end so that the bending planes of all flexible tendon-driven inner tubes 14 are aligned with a given two-dimensional target plane.

To navigate the robot 10, the user inserts the rigid straight outer tube 12 into a portion of patient's brain 38 using imaging guidance. When the rigid straight outer tube 12 is in place proximate the target tissue 40, which may be near various blood vessels 42, the user deploys all tendon-driven inner tubes 14 together, advancing them distally. Then the robot's tendon steering mechanism is used to hold the innermost tendon-driven inner tube in place while advancing the remaining tendon-driven inner tubes 14 and adjusting the tension of the tubes as needed based on the reference trajectory. Next, the two innermost tubes tendon-driven inner tubes are held in place while the remaining tendon-driven inner tubes are advanced and the tension is adjusted. This process is repeated until the outermost tendon-driven inner tube is deployed.

Once the distal tip of the robot 10 reaches a target tissue 40, the laser is activated to ablate the target tissue in the ablation region 44. Afterwards, the robot 10 is retracted by retracting the tendon-driven inner tubes 14 in the reverse sequence (e.g., the outermost tendon-driven inner tube is retracted onto the second-outermost tendon-driven inner tube, then the outermost and second-outermost tendon-driven inner tubes are retracted together onto the third-outermost tendon-driven inner tube, and so on until the innermost tendon-driven inner tube is retracted). When the number of tendon-driven inner tubes is larger than the number of arcs of the reference trajectory, the redundant inner tendon-driven inner tubes 14 stay inside the straight rigid outer tube 12. When retraction is complete, the user can then rotate the robot navigation plane, thereby moving the robot to other target locations and ablating tissue in the same manner.

Throughout the deployment and retraction processes, the deployment force of the tendon wires 16 for each tendon-driven inner tube 14 is regulated to allow the robot to move along each arc of the curvilinear reference trajectory, which is determined based, at least in part, on user input. To determine the deployment force of the tendon wires 16, a database of measured curvatures for the length of at least one robot segment (e.g., proximal segment, middle segment, and distal segment of the fully deployed robot) is used. The database can be pre-generated by measuring the curvature of each robot segment length and the corresponding deployment force in soft tissues. The robot processor searches the database for curvatures (for each robot segment) that are close to the arc of the reference trajectory and their corresponding deployment forces. Linear interpolation among the determined deployment forces is then performed to compute the required tendon wire forces for each tendon-driven inner tube 14.

### Various Notes

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents and patent applications are incorporated herein by reference. In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel,” “perpendicular,” “round,” or “square,” are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

As used herein, the term “about,” when referring to a value is meant to encompass variations of, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A steerable robotic device comprising:
a rigid straight outer tube;
at least one telescopic flexible tendon-driven inner tube, including an outermost telescopic flexible tendon-driven inner tube; and
an optical fiber extending through the at least one telescopic flexible tendon-driven inner tube, the optical fiber coupled distally to a distal tip of the outermost telescopic flexible tendon-driven inner tube and proximally to a laser generator.

2. The steerable robotic device of claim 1, wherein the at least one telescopic flexible tendon-driven inner tube comprises a distal segment having notches extending longitudinally along a portion of a wall of the inner tube.

3. The steerable robotic device of claim 2, wherein the at least one telescopic flexible tendon-driven inner tube is configured to bend along a curved trajectory.

4. The steerable robotic device of claim 1, further comprising a flexible sleeve covering the outermost telescopic flexible tendon-driven inner tube.

5. The steerable robotic device of claim 1, wherein the rigid straight outer tube and the at least one telescopic flexible tendon-driven inner tube comprise superelastic nitinol.

6. The steerable robotic device of claim 1, further comprising a plurality of nonmagnetic rotary or linear actuators.

7. The steerable robotic device of claim 1, comprising an inner telescopic flexible tendon-driven inner tube, wherein a distal tip of the inner flexible tendon-driven inner tube is configured to remain inside the outermost telescopic flexible tendon-driven inner tube.

8. The steerable robotic device of claim 1, further comprising a processor configured to determine a deployment force of the at least one telescopic flexible tendon-driven inner tube based on a reference trajectory of the at least one telescopic flexible tendon-driven inner tube.

9. The steerable robotic device of claim 8, wherein the processor is configured to determine the deployment force of the at least one telescopic flexible tendon-driven inner tube using a database of measured curvatures corresponding to one or more robot segment lengths.

10. The steerable robotic device of claim 9, wherein the database of measured curvatures is generated based a measured soft-tissue deployment force of the one or more robot segment lengths.

11. The steerable robotic device of claim 9, wherein the processor performs linear interpolation among the determined deployment forces to compute a tendon wire force for each telescopic flexible tendon-driven inner tube.

12. The steerable robotic device of claim 1, wherein the rigid straight outer tube has an outer diameter of about 2.2 mm or smaller.

13. The steerable robotic device of claim 1, further comprising a FBG optical fiber extending through the at least one telescopic flexible tendon-driven inner tube, the FBG optical fiber including Fiber Bragg gratings (FBGs).

14. The steerable robotic device of claim 10, wherein the FBG optical fiber including FBGs is embedded in a silicone polymer cylinder hosting photothermal nanoparticles, the silicone polymer cylinder located within the distal tip of the outer telescopic flexible tendon-driven inner tube.

15. The steerable robotic device of claim 10, wherein the FBG optical fiber including FBGs is surrounded by a flexible saline cooling tube extending through the at least one telescopic flexible tendon-driven inner tube.

16. The steerable robotic device of claim 10, wherein the FBGs is configured to monitor temperatures changes at the distal tip of the outer telescopic flexible tendon-driven inner tube.

17. A method of ablating a target tissue using the steerable robotic device of claim 1, the method comprising:
inserting the rigid straight outer tube into the target tissue;
rotating the at least one telescopic flexible tendon-driven inner tube to define a first navigation plane;
deploying the at least one telescopic flexible tendon-driven inner tube;
adjusting a tendon wire to control a curved trajectory of the at least one telescopic flexible tendon-driven inner tube;
ablating the target tissue; and
retracting the at least one telescopic flexible tendon-driven inner tube.

18. The method of claim 17, wherein the steerable robotic device comprises an inner telescopic flexible tendon-driven inner tube positioned inside the outer telescopic flexible tendon-driven inner tube, and wherein the deploying step comprises:
distally advancing the inner and outer telescopic flexible tendon-driven inner tubes together; and
while holding the inner telescopic flexible tendon-driven inner tube in place, distally advancing the outer flexible tendon-driven inner tube.

19. The method of claim 17, wherein the retracting step comprises:
proximally retracting the outer telescopic flexible tendon-driven inner tube onto the inner telescopic flexible tendon-driven inner tube; and
after the outer telescopic flexible tendon-driven inner tube is retracted onto the inner telescopic flexible tendon-driven inner tube, proximally retracting the inner and outer telescopic flexible tendon-driven inner tubes into the rigid straight outer tube.

20. The method of claim 17, further comprising rotating the at least one telescopic flexible tendon-driven inner tube to define a second navigation plane.
